(19) United States
(12) Patent Application Publication
(10) Pub. No.: US 2011/0224505 A1
(43) Pub. Date: Sep. 15, 2011

(54) USER WEARABLE PORTABLE COMMUNICATIVE DEVICE

(76) Inventor: RAJENDRA PADMA SADHU, Somerset, NJ (US)

(21) Appl. No.: 12/722,554

(22) Filed: Mar. 12, 2010

(51) Int. Cl. A61B 5/00; G01S 19/42; G08B 1/08

(52) U.S. Cl. 600/301; 342/357.25; 340/539.12

(57) ABSTRACT

A user wearable portable communicative device and a method for establishing a communication between a remote health care monitoring center and a portable communicative device are disclosed. The portable communicative device includes a location tracking module for tracking a current location of the user through a communication network, a plurality of user controllable switches for enabling the user to establish a voice based communicative interaction with a plurality of health care providers present over a remote health care monitoring center, a physiological data collecting module for collecting the physiological data from a plurality of physiological data collecting devices of the user, a motion detecting module for reducing an amount of power consumed by the portable communicative device and a plurality of reminder modules for alerting the user to get a medication at a predetermined time.

### TECHNICAL FIELD OF THE INVENTION

The present invention generally relates to the field of communication devices. More particularly the present invention relates to a user wearable portable communicative device.

### BACKGROUND OF THE INVENTION

Generally, the number of users requiring the remote health care are increasing in the past few years. The remote health care systems use a variety of health care devices to continuously monitor the user. But these types of health care devices are not portable and restricted to one geographical area.

Conventionally, the panic button type devices interlink the user to the emergency response team via landline or mobile telephone. If the user is suddenly disabled during a sudden health crisis, such as in a heart attack or a serious fall situation, the panic-button type devices become useless. Further more if the person is able to press the button the person should be within the effective wireless transmission distance to the device that dials the telephone to report the emergency. The panic button type devices are not effective for the users when the users are in unconscious state or rendered incapacitated by a fall or other medical condition. Furthermore no vital information on the user's status like heart rate, blood pressure, breath rate, body temperature, oxygen level and the like will be transmitted to the response team to provide further medical assistance. Consequently, it is difficult for the response team to diagnose and provide treatment to the user.

Typically, the health monitoring systems should have an adjustable emergency alert level throughout a day for different levels of activity. The capacity to determine the location of the person, who requires immediate medical assistance, should be more effective, accurate and relevant to provide proper medical assistance. The global positioning systems (GPS) determine the location of the user when signals from multiple GPS satellites are received by the system. The GPS system fails to determine the location of the user when signals are not received from more than one satellite due to shielding by buildings or geographic features or improper antenna orientation. The conventional GPS tracking systems comprises of multiple integrated circuit chips. Consequently the multiple integrated circuit chips consume more power compared to the single integrated circuit chips with the combined capabilities of the individual integrated circuit chips. Furthermore the conventional GPS receivers can determine the location of the user when there is clear view of the sky within several feet of the GPS receiver's antenna.

Typically, many health care facilities perform the vital sign monitoring of the user only once in a week due to the time and money needed to perform these operations. If the user's vital signs are checked only once in a week, the declining health condition of the user is detected after the health condition is worsened. Furthermore this is eliminating the opportunity for early intervention. The user's physiological parameters such as pulse rate, heart beat rate, electrocardiogram (EKG), blood pressure, breathing rate, body temperature and the like should be measured continuously. Further the measured parameters and the location of the user should be transmitted to the central monitoring system to continuously monitor the physiological parameters of the user. When there is a sudden abnormal change in the user's physiological parameters then the control center will perform the further operation.

Hence there is a need for a user wearable portable communication device for tracking the location of the user and to further communicate with a remote health care monitoring center.

### BRIEF SUMMARY OF THE INVENTION

A user wearable portable communicative device and a method for establishing a communication between a remote health care monitoring center and a portable communicative device are disclosed. According to a first aspect, the portable communicative device includes a location tracking module for tracking a current location of the user through a communication network. The device includes a location tracking module for tracking the current location of the user through at least one of an assisted global positioning system and a global positioning system.

According to the first aspect, the portable communicative device includes a plurality of user controllable switches for enabling the user to establish a voice based communicative interaction with a plurality of health care providers present over a remote health care monitoring center. The communication between the wearable portable communicative device and the remote health care monitoring center is enabled through a communication network consisting at least one of a global system for mobile communications, a general packet radio service and the like.

According to the first aspect, the portable communicative device includes a physiological data collecting module for collecting the physiological data from a plurality of physiological data collecting devices of the user. The plurality of physiological data collecting devices transmits the physiological data to the portable communicative device over a short range communication network employing a set of communication protocols. The set of communication protocols consisting at least one of a transmission control protocol/internet protocol, a hyper text transfer protocol and the like. The short range communication network includes at least one of a bluetooth network, a zigbee network and the like. The plurality of physiological data collecting devices comprising at least one of a chest belt, a blood pressure monitor, a body fat and weight scale, a blood sugar monitor and the like. The chest belt is adapted to utilize as at least one of a heart rate reader, a pulse rate reader, an electrocardiogram reader, a pacemaker reader, a body temperature reader and the like.

According to the first aspect, the portable communicative device includes a motion detecting module for reducing an amount of power consumed by the portable communicative device.

According to the first aspect, the portable communicative device includes a plurality of reminder modules for alerting the user to get a medication at a predetermined time.

According to the first aspect, the portable communicative device further includes a universal serial bus provision for receiving the physiological data from the plurality of physiological data collecting devices.

According to a second aspect, a method for establishing a communication between a remote health care monitoring center and a portable communicative device is disclosed. The method for establishing a communication with a remote health care monitoring center with a portable communicative device includes collecting a physiological data from a plurality of physiological data collecting devices of the user with a physiological data collecting module.

According to the second aspect, the method for establishing a communication between a remote health care monitoring center and a portable communicative device includes transmitting the collected physiological data to a remote health care monitoring center over a communication network.

According to the second aspect, the method for establishing a communication between a remote health care monitoring center and a portable communicative device includes enabling a voice based communicative interaction between the user and a plurality of health care providers present over the remote health care monitoring center by using a plurality of user controllable switches.

According to the second aspect, the method for establishing a communication between a remote health care monitoring center and a portable communicative device includes tracking a current location of the user with a location tracking module.

According to the second aspect, the method for establishing a communication between a remote health care monitoring center and a portable communicative device further includes reducing an amount of power consumed by the portable communicative device with a motion detecting module with a plurality of motion detecting sensors.

According to the second aspect, the method for establishing a communication between a remote health care monitoring center and a portable communicative device further includes alerting the user to take a medication at a predetermined time by using a plurality of reminder modules.

According to the second aspect, the method for establishing a communication between a remote health care monitoring center and a portable communicative device further includes employing a set of communication protocols for enabling a communication between the plurality of physiological data collecting devices and the portable communicative device.

### BRIEF DESCRIPTION OF THE DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is diagram depicting a user wearable portable communicative device.

FIG. 2 is a diagram depicting a user wearable portable communicative device serving as a pendent.

FIG. 3 is a diagram depicting a front view of a user wearable portable communicative device.

FIG. 4 is a diagram depicting a side view of a user wearable portable communicative device.

FIG. 5 is a diagram depicting a system for enabling a communication between a plurality of physiological data collecting devices and a remote health care monitoring center.

### DETAILED DESCRIPTION OF THE INVENTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

Exemplary embodiments of the present invention are directed towards a user wearable portable communicative device and a method for establishing a communication between a remote health care monitoring center and a portable communicative device are disclosed. According to a first aspect, the portable communicative device includes a location tracking module for tracking a current location of the user through a communication network. The device includes location tracking module for tracking the current location of the user through at least one of an assisted global positioning system and a global positioning system.

According to the first aspect, the portable communicative device includes a plurality of user controllable switches for enabling the user to establish a voice based communicative interaction with a plurality of health care providers present over a remote health care monitoring center. The communication between the wearable portable communicative device and the remote health care monitoring center is enabled through a communication network consisting at least one of a global system for mobile communications, a general packet radio service and the like.

According to the first aspect, the portable communicative device includes a physiological data collecting module for collecting the physiological data from a plurality of physiological data collecting devices of the user. The plurality of physiological data collecting devices transmits the physiological data to the portable communicative device over a short range communication network employing a set of communication protocols. The set of communication protocols consisting at least one of a transmission control protocol/internet protocol, a hyper text transfer protocol and the like. The short range communication network includes at least one of a bluetooth network, a zigbee network and the like. The plurality of physiological data collecting devices comprising at least one of a chest belt, a blood pressure monitor, a body fat and weight scale, a blood sugar monitor and the like. The chest belt is adapted to utilize as at least one of a heart rate reader, a pulse rate reader, an electrocardiogram reader, a pacemaker reader, a body temperature reader and the like.

According to the first aspect, the portable communicative device includes a motion detecting module for reducing an amount of power consumed by the portable communicative device.

According to the first aspect, the portable communicative device includes a plurality of reminder modules for alerting the user to get a medication at a predetermined time.

According to the first aspect, the portable communicative device further includes a universal serial bus provision for receiving the physiological data from the plurality of physiological data collecting devices.

According to a second aspect, a method for establishing a communication between a remote health care monitoring center and a portable communicative device is disclosed. The method for establishing a communication with a remote health care monitoring center with a portable communicative device includes collecting a physiological data from a plurality of physiological data collecting devices of the user with a physiological data collecting module.

According to the second aspect, the method for establishing a communication between a remote health care monitoring center and a portable communicative device includes transmitting the collected physiological data to a remote health care monitoring center over a communication network.

According to the second aspect, the method for establishing a communication between a remote health care monitoring center and a portable communicative device includes enabling a voice based communicative interaction between the user and a plurality of health care providers present over the remote health care monitoring center by using a plurality of user controllable switches.

According to the second aspect, the method for establishing a communication between a remote health care monitoring center and a portable communicative device includes tracking a current location of the user with a location tracking module.

According to the second aspect, the method for establishing a communication between a remote health care monitoring center and a portable communicative device further includes reducing an amount of power consumed by the portable communicative device with a motion detecting module with a plurality of motion detecting sensors.

According to the second aspect, the method for establishing a communication between a remote health care monitoring center and a portable communicative device further includes alerting the user to take a medication at a predetermined time by using a plurality of reminder modules.

According to the second aspect, the method for establishing a communication between a remote health care monitoring center and a portable communicative device further includes employing a set of communication protocols for enabling a communication between the plurality of physiological data collecting devices and the portable communicative device.

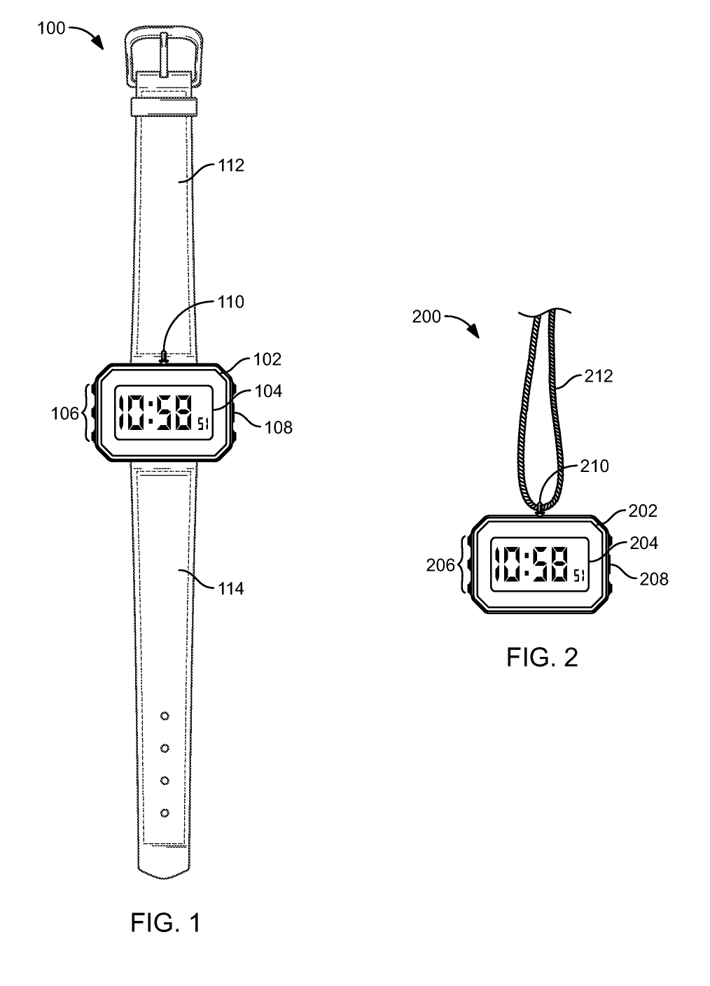

Referring to FIG. 1 is a diagram 100 depicting a user wearable portable communicative device. The portable communicative device includes a main housing 102, a display module 104, a plurality of user controllable switches 106, a universal serial bus provision 108, a first strap 112, a second strap 114. The first strap 112 is attached to the first end of the main housing 102 and the second strap 114 is attached to the second end of the main housing 102. The main housing 102 includes a display module 104 for displaying the data. The display module is an organic light emitting diode (OLED) made with organic material for displaying the data, according to a non limiting exemplary embodiment of the present invention. The main housing 102 includes a universal serial bus provision 108 for receiving the physiological data from the plurality of physiological data collecting devices. Further the physiological data from the plurality of physiological data collecting devices is transmitted to the portable communicative device through a plurality of short range communication networks, wherein the plurality of short range communication networks includes a blue tooth network, a zigbee network and the like. The received physiological data is further transmitted from the portable communicative device to the remote health care monitoring center through a communication network. The communication network includes at least one of a global system for mobile communications (GSM), a general packet radio service (GPRS) and the like. The global system for mobile communications further includes dual band and quad band for supporting multiple frequencies of data transmission. The main housing 102 further includes a plurality of user controllable switches 106 for enabling the user to establish a voice based communicative interaction with a plurality of health care providers present over a remote health care monitoring center. A holed provision 110 fastened to the main housing 102 allows the user to wear the portable communicative device as a pendent. Further the location of the user is tracked by the location tracking module embedded in the main housing 102 of the portable communicative device.

Referring to FIG. 2 is diagram 200 depicting a user wearable portable communicative device serving as pendent. The user wearable portable communicative device includes a main housing 202, an OLED display module 204 with organic material for displaying the data, a plurality of user controllable switches 206 enabling the user to establish a voice based communication with the remote health care monitoring center, a universal serial bus provision 208 for receiving the physiological data from the plurality of physiological data collecting devices and a holed provision 210 for inserting a chain 212 into the provision to allow the user to wear the portable communicative device as a pendent. The motion detecting module embedded in the main housing 202 of the portable communicative device reduces the power consumed by the portable communication device.

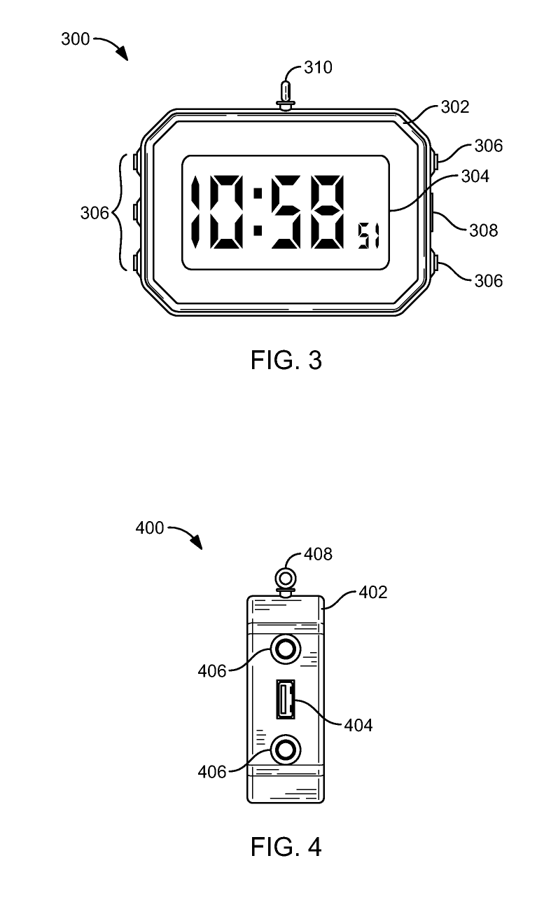

Referring to FIG. 3 is a diagram 300 depicting a front view of a user wearable communicative device. The physiological data collecting module of the portable communicative device serves as an interface between the plurality of physiological data collecting devices and the portable communicative device. An OLED display 304 made with an organic material present over a portable communicative device displays the time and other data. The universal serial bus provision 308 enables the user to receive the data from the plurality of physiological data collecting devices through a short range communication network employing a set of communication protocols. The set of communication protocols include a transmission control protocol/internet protocol, hyper text transfer protocol and the like. The plurality of user controllable switches 306 fastened to the main housing 302 of the portable communicative device allows the plurality of users to communicate with the remote health care monitoring center for receiving the emergency medical support. The motion detecting module embedded in the main housing 302 reduces the amount of power consumed by the portable communicative device 300 and the location tracking module tracks the current location of the user. The physiological data collecting module collects the physiological data from the physiological collecting devices and transmits the collected physiological data to the remote health care monitoring center over the communication network. The medicine reminder presented on the portable communicative device alerts the user to take proper medicine at the predetermined time.

Referring FIG. 4 is diagram 400 depicting a side view of a user wearable portable communicative device. According to a non limiting exemplary embodiment of the present invention, the portable communicative device 400 includes a main housing 402 and a provision 404 for receiving the data from the plurality of physiological data collecting devices. The provision 404 is a universal serial bus provision. A plurality of user controllable switches 406 are attached to the main housing 402 of the portable communicative device 400. The plurality of user controllable switches 406 establishes a voice based communicative interaction between the portable communicative device and the remote health care monitoring center over the communication network.

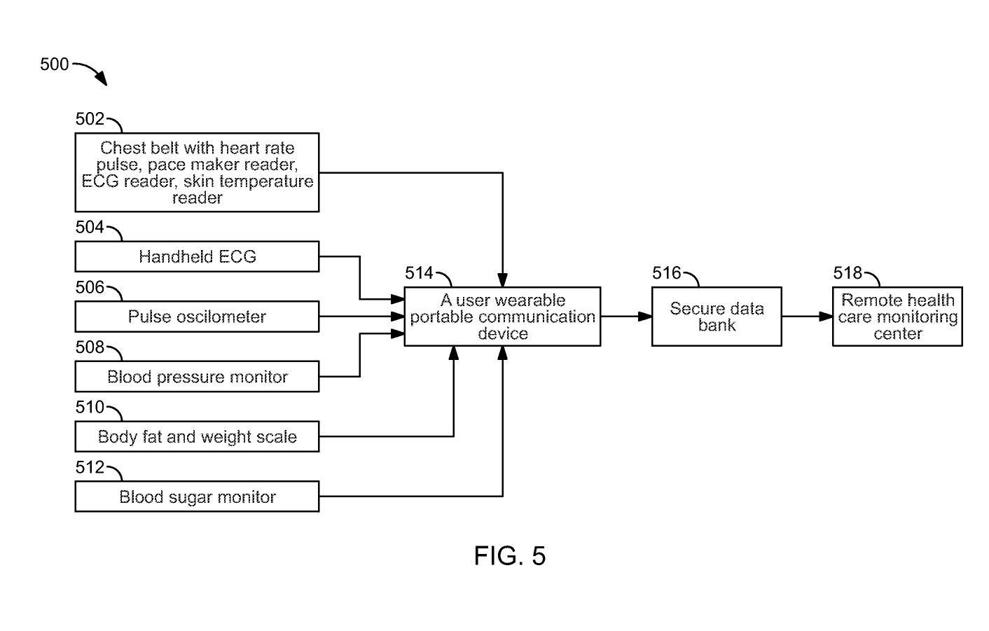

Referring to FIG. 5 is a diagram 500 depicting a system for enabling a communication between a plurality of physiological data collecting devices and a remote health care monitoring center. The system for enabling a communication between a plurality of physiological data collecting devices and a remote health care monitoring center includes the plurality of physiological data collecting devices. The data collecting devices include a chest belt 502, a handheld electrocardiogram 504, a pulse oscilometer 506, a blood pressure monitor 508, a body fat and weight scale 510, a blood sugar monitor 512 and the like. The plurality of physiological data collecting devices communicates with the user wearable portable communicative device 514 through the short range communication network. The collected physiological data from the plurality of physiological data collecting devices is transmitted from the user wearable portable communicative device 514 to the remote health care monitoring center 518 through the communication network and the collected physiological data is stored in the secured data bank 516. The communication network includes a global system for mobile communications (GSM), a general packet radio service (GPRS) and the like.

According to a non limiting exemplary embodiment of the present invention, the plurality of physiological data collecting devices includes the chest belt 502 for collecting a heart rate, a pulse rate, an electrocardiogram readings, a body temperature rate and the like. The chest belt further serves as a pace maker reader. The hand held electrocardiogram 504 is used to collect the electrocardiogram readings of the user and the pulse oscilometer 506 is used to collect the pulse rate of the user. According to an exemplary embodiment of the present invention the blood pressure monitor 508 collects the blood pressure readings of the user and the body fat and weight scale measures the user's body fat and weight. Further the blood sugar monitor determines the sugar level in the blood of the user.

According to an exemplary embodiment of the present invention, the portable communicative device 514 collects the physiological data from the plurality of physiological data collecting devices through the short range communication network like blue tooth network and zigbee network. Further the received physiological data is transmitted to the remote health care monitoring center 518 through a communication network.

According to an exemplary embodiment of the present invention, the portable communicative device further includes a location tracking module for tracking the current location of the user to provide medical assistance. The physiological data collecting module collects the physiological data from the plurality of physiological devices. Further a motion detecting module used to reduce the power consumed by the portable communicative device. The portable device further includes a plurality of reminders to alert the user to take proper medication at predetermined time.

As will be appreciated by a person skilled in the art the present invention provides a variety of advantages. Firstly, the invention provides a location tracking module for tracking the current location of the user. Secondly, the invention provides a remote monitoring of physiological data by the plurality of health care providers present over the remote health care monitoring center. Thirdly the invention provides a voice based communicative interaction between the portable communicative device and the remote health care monitoring center. Fourthly the invention provides storage of the physiological data in a secure data bank.

While specific embodiments of the invention have been shown and described in detail to illustrate the inventive principles, it will be understood that the invention may be embodied otherwise without departing from such principles.

1. A user wearable portable communicative device, comprising:
a location tracking module for tracking a current location of the user through a communication network;
a plurality of user controllable switches for enabling the user to establish a voice based communicative interaction with a plurality of health care providers present over a remote health care monitoring center;
a physiological data collecting module for collecting the physiological data from a plurality of physiological data collecting devices of the user;
a motion detecting module for reducing an amount of power consumed by the portable communicative device; and
a plurality of reminder modules for alerting the user to get a medication at a predetermined time.

2. The portable communicative device of claim 1, wherein the location tracking module for tracking the current location of the user through at least one of:
an assisted global positioning system; and
a global positioning system.

3. The portable communicative device of claim 1, wherein the plurality of physiological data collecting devices transmit the physiological data to the portable communicative device over a short range communication network employing a set of communication protocols.

4. The portable device of claim 3, wherein the set of communication protocols comprising at least one of:
a transmission control protocol/internet protocol; and
a hyper text transfer protocol.

5. The portable communication device of claim 3, wherein the short range communication network comprising at least one of:
a bluetooth network; and
a zigbee network;

6. The portable communicative device of claim 1 further comprising a universal serial bus provision for receiving the physiological data from the plurality of physiological data collecting devices.

7. The portable communicative device of claim 1, wherein the plurality of physiological data collecting devices comprising at least one of a chest belt;
a blood pressure monitor;
a body fat and weight scale; and
a blood sugar monitor.

8. The portable communicative device of claim 7, wherein the chest belt is adapted to utilize as at least one of:
a heart rate reader;
a pulse rate reader;
an electrocardiogram reader;
a pacemaker reader; and
a body temperature reader.

9. The user wearable portable communicative device of claim 1, wherein the communication between the wearable portable communicative device and the remote health care monitoring center is enabled through a communication network comprising at least one of:
a global system for mobile communications; and
a general packet radio service.

10. A method for establishing a communication between a remote health care monitoring center and a portable communicative device, comprising:
collecting a physiological data from a plurality of physiological data collecting devices of the user with a physiological data collecting module;
transmitting the collected physiological data to a remote health care monitoring center over a communication network;
enabling a voice based communicative interaction between the user and a plurality of health care providers present over the remote health care monitoring center by using a plurality of user controllable switches; and
tracking a current location of the user with a location tracking module.

11. The method of claim 9 further comprising reducing an amount of power consumed by the portable communicative device with a motion detecting module with a plurality of motion detecting sensors.

12. The method of claim 9 further comprises alerting the user to take a medication at a predetermined time by using a plurality of reminder modules.

13. The method of claim 9 further comprising employing a set of communication protocols for enabling a communication between the plurality of physiological data collecting devices and the portable communicative device.
